(19) United States
(12) Patent Application Publication
(10) Pub. No.: US 2023/0393397 A1
(43) Pub. Date: Dec. 7, 2023

(54) HEAD-MOUNTED DEVICE USING A BIOSENSOR SIGNAL FOR POWER MANAGEMENT

(71) Applicant: Hewlett-Packard Development Company, L.P., Spring, TX (US)

(72) Inventors: David Steven Daley, Taipei City (TW); Yih-Lun HUANG, Taipei (TW); Ling I HUNG, Taipei (TW)

(21) Appl. No.: 18/249,161

(22) PCT Filed: Oct. 15, 2020
(86) PCT No.: PCT/US2020/055803

§ 371 Date: Apr. 14, 2023

(51) Int. Cl. G02B 27/01; G02B 27/00; G06F 3/01; G06F 1/3231; G06F 1/3234

(52) U.S. Cl. CPC G02B 27/017; G02B 27/0093; G06F 3/015; G06F 1/3231; G06F 1/3275; G02B 2027/014

(57) ABSTRACT

An example head-mounted device includes: a frame to nest the device on a head of a user; a biosensor disposed on the frame, the biosensor to: detect a biological characteristic of the user; generate a signal representing the biological characteristic of the user; and a power management controller coupled to the biosensor, the controller to, when the signal corresponds to a predefined profile, determine that the head-mounted device is in a transitional state and change a power state of the head-mounted device based on the transitional state.

### BACKGROUND

Head-mounted devices, such as extended reality display systems, consume power when operational, and hence may include power management systems to allow the devices to be turned on when in use and off to conserve energy when not in use.

### BRIEF DESCRIPTION OF THE DRAWINGS

FIG. 1 is a schematic of an example head-mounted device using biosensor signals for power management.

FIG. 2 is a block diagram of another example head-mounted device using biosensor signals for power management.

FIGS. 3A and 3B are diagrams of example biosensor signals.

FIG. 4 is a flowchart of an example method of managing power based on a biosensor signal in the head-mounted device of FIG. 2.

FIG. 5 is a block diagram of another example head-mounted device using multiple biosensor signals for power management.

FIG. 6 is a flowchart of an example method of managing power based on multiple biosensor signals in the head-mounted device of FIG. 5.

### DETAILED DESCRIPTION

The power management systems of head-mounted devices may be manual and thus involve a user actively turning the device on or off. This may be cumbersome for the user and may waste energy if the user forgets to turn the device off after use. Some solutions may use presence sensors to detect or predict when a user is nearby and oriented towards the device but such presence sensors may be foiled by nearby objects or triggered while in carrying cases. Other solutions may use biosensors to detect when the device is mounted on the user's head, and subsequently transitioning from a low power mode to an operating mode but such systems may create a perceived lag and dissatisfaction from the user as the device powers on when the device is properly mounted.

An example head-mounted device may use biosensors to detect a biological characteristic of a user and generate a signal not only when the user is actively using the device (i.e., when the device is in physical contact with the user's head), but also when the head-mounted device is near the user. In particular, the biosensors generate signals with specific profiles when the head-mounted device is in a transitional state, moving towards the head of the user (i.e., when the user is putting the head-mounted device on) or moving away from the head of the user (i.e., when the user is taking the head-mounted device off). A power management controller of the head-mounted device may compare the signal received from the biosensor to the predefined profiles, and, when the signal corresponds to the predefined profile, determine that the head-mounted device is in a transitional state and change the power state of the head-mounted device accordingly.

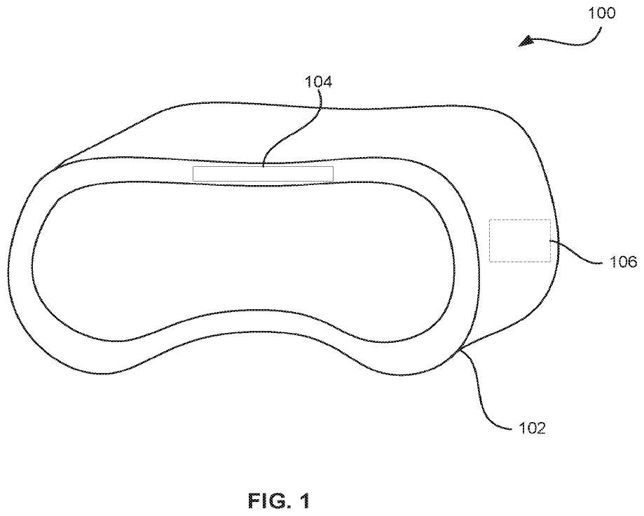

FIG. 1 shows a block diagram of an example head-mounted device 100 with an integrated biosensor for power management. The head-mounted device 100 (also referred to herein as simply the device 100) includes a frame 102 to nest the device 100 on or against the head of a user, a biosensor 104 to detect a biological characteristic of the user and generate a signal representing the biological characteristic of the user, and a power management controller 106 coupled to the biosensor to manage the power state of the device 100 based on the signals from the biosensor 104.

The device 100 may be a head-mounted display device, such as a virtual reality, augmented reality, mixed reality or other extended reality display device. In other examples, the device 100 may be another head-mounted device including sensing or monitoring headband, an audio headset, or the like. Generally, the device 100 includes components which consume power, such as a processor, a display, a communications interface such as a transceiver, or the like. The frame 102 is to support or otherwise nest the device 100 on or against the head of the user. For example, the frame 102 may include a contact portion to interface with the user's face or eyes (e.g., against which the user may set their face or eyes), a band or strap to support the device 100 on the user's head, arms or temples including tips to rest on the user's ears, or other similar components. The frame 102 may further support the other components of the device 100, including a display device, lenses, and the like.

The biosensor 104 is a sensor which detects a biological characteristic of the user and generates a signal representing the biological characteristic. For example, the biosensor 104 may detect muscle movement, facial movement or features, heart rate or pulse, and the like. The biosensor 104 may be, for example, a photoplethysmography (PPG) sensor to detect a change in blood circulation of the user, an electromyography (EMG) sensor to detect electrical activity of facial muscles of the user, an eye-tracking sensor, a mouth camera, a capacitive sensor, an ultrasound sensor, or the like. In particular, the biosensor 104 is to detect characteristics which are unique to human users, and which does not trigger a discernable signal difference by inanimate objects which may be near the device 100. Further, the biosensor 104 is selected to detect the biological characteristic of a nearby user, rather than generating a signal strictly when the biosensor 104 has appropriate physical contact with the user for normal use of the biosensor 104. In the present example, the biosensor 104 is integrated with the head-mounted device 100 and disposed on the frame 102.

The power management controller 106 (also referred to herein as simply the controller 106) may include a central processing unit (CPU), a microcontroller, a microprocessor, a processing core, a field-programmable gate array (FPGA), or similar device capable of executing machine-readable instructions. The controller 106 may cooperate with a memory to execute instructions. Memory may include a non-transitory machine-readable storage medium that may be may electronic, magnetic, optical, or other physical storage device that stores executable instructions. The machine-readable storage medium may include, for example, random access memory (RAM), read-only memory (ROM), electrically-erasable programmable read-only memory (EEPROM), flash memory, a storage drive, an optical disc, and the like. The machine-readable storage medium may be encoded with executable instructions.

Generally, the controller 106 manages the power states of the device 100, and in particular, of power consuming components of the device 100, such as a display, a transceiver or other communications interface, a main processor, or similar. More particularly, the controller 106 manages the power states of the device 100 based on signals from the biosensor 104. That is, when a signal received from the biosensor 104 corresponds to a predefined profile, the controller 106 determines that the device 100 is in a transitional state and changes a power state of the device 100 based on the transitional state.

The predefined profile may define various threshold values, such as a threshold peak amplitude of the signal (i.e., a threshold value of the absolute value of the signal), or a threshold peak-to-peak amplitude of a wave of the signal (i.e., the amplitude between adjacent high and low peaks of the wave of the signal). The predefined profile may also be defined based on a length of time the signal remains above or below the threshold values, and other similar factors.

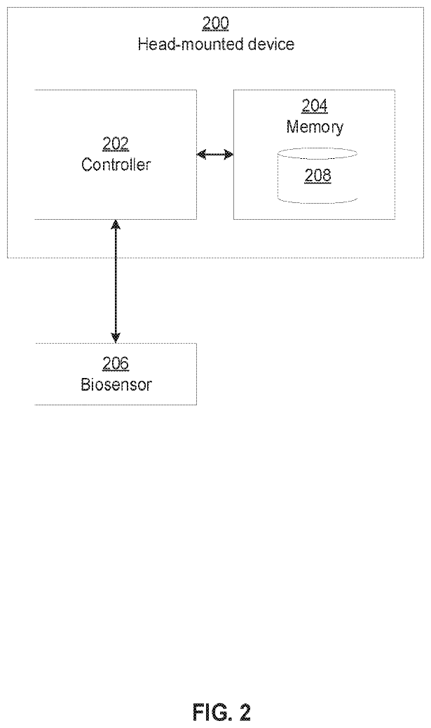

Referring to FIG. 2, a block diagram of certain internal components of another example head-mounted device 200 is depicted. The device 200 is similar to the device 100, and may be a head-mounted display device, such as an extended reality display device, or another head-mounted device. The device 200 includes a controller 202 and a memory 204 and is further associated with a biosensor 206.

The controller 202 is a power management controller and may include a central processing unit (CPU), a microcontroller, a microprocessor, a processing core, a field-programmable gate array (FPGA), or similar device capable of executing machine-readable instructions. The controller 202 may also include or be interconnected with a non-transitory machine-readable storage medium, such as the memory 204, that may be electronic, magnetic, optical, or other physical storage device that stores executable instructions. In particular, the memory 204 may store an application for managing the power states of the device 200 based on signals from the biosensor 206.

Accordingly, the controller 202 is also in communication with the biosensor 206. The biosensor 206 is a sensor which detects a biological characteristic of the user and generates a signal representing the biological characteristic. For example, the biosensor 206 may detect muscle movement, facial movement or features, heart rate or pulse, and the like. The biosensor 206 may be, for example, a PPG sensor to detect a change in blood circulation of the user, an EMG sensor to detect electrical activity of facial muscles of the user, an eye-tracking sensor, a mouth camera, a capacitive sensor, an ultrasound sensor, or the like. In particular, the biosensor 206 is to detect characteristics which are unique to human users, and which does not trigger a discernable signal difference by inanimate objects which may be near the biosensor 206. The biosensor 206 may be integrated with the head-mounted device 200, or may be independent from the head-mounted device 200. For example, a PPG sensor may be integrated in a wristband or glove associated with the head-mounted device 200 (e.g., a hand or other accessory for use with the head-mounted device 200 in a virtual reality system).

The memory 204 further stores a signal profile repository 208 tracking associations between signal profiles of signals detected by the biosensor 206 and activity states of the device 200. The activity states of the head-mounted device 200 track the positionally-based state of use of the device 200 rather than the power states of the device 200, which track the functional status of the operative components of the device 200.

In particular, the device 200 has an active state, in which the device 200 is in place on a user's head, in a ready-to-use positional configuration. When the device 200 is in the active state, the biosensor 206 detects an active signal profile. For example, the biosensor 206 may detect a signal having a peak amplitude above a predefined active threshold value. In other examples, a peak-to-peak amplitude of the signal may also contribute to the active signal profile. For example, in addition to or instead of detecting a signal having a peak amplitude above the predefined active threshold value, the signal detected by the biosensor 206 may have a peak-to-peak amplitude below a second predefined active threshold value. That is, when a wave of the signal has a relatively small peak-to-peak amplitude, the signal may be determined to correspond with the active signal profile. In still further examples, the signal may correspond to the active signal profile when its peak amplitude is below the active threshold value, or when its peak-to-peak amplitude is above the second active threshold value.

The device 200 further has an inactive state, in which the device 200 is removed from a user's head. For example, the device 200 may be resting on a surface, or packed in a case. When the device 200 is in the inactive state, the biosensor 206 detects an inactive signal profile. For example, the biosensor 206 may detect a signal which has a peak amplitude below predefined inactive threshold value. In other examples, a peak-to-peak amplitude of the signal may also contribute to the inactive signal profile. For example, in addition to or instead of detecting a signal having a peak amplitude below the predefined inactive threshold value, the signal detected by the biosensor 206 may have a peak-to-peak amplitude above a second predefined inactive threshold value. That is, when a wave of the signal has a relatively large peak-to-peak amplitude, the signal may be determined to correspond with the inactive signal profile. In still further examples, the signal may correspond to the inactive signal profile when its peak amplitude is above the inactive threshold value, or when its peak-to-peak amplitude is below the second inactive threshold value.

The device 200 also has a first transitional state, in which the device 200 is being transitioned or moved from the inactive state to the active state. For example, a user of the device 200 may be in the process of putting the device 200 on (e.g., mounting the head-mounted device 200 on the user's head). The first transitional state corresponds to a first transitional signal profile detected by the biosensor 206. For example, the first transitional signal profile may be defined by the movement of the peak amplitude of the signal from below the inactive threshold value to above the inactive threshold value. In some examples, a peak-to-peak amplitude of the signal may contribute to the first transitional signal profile.

The device 200 further has a second transitional state, in which the device 200 is being transitioned or moved from the active state to the inactive state. For example, a user of the device 200 may be in the process of removing the device 200 (e.g., taking the head-mounted device 200 off of the user's head). The second transitional state corresponds to a second transitional signal profile detected by the biosensor 206. For example, the second transitional signal profile may be defined by the movement of the peak amplitude of the signal from above the active threshold value to below the active threshold value. In some examples, a peak-to-peak amplitude of the signal and a length of time the signal remains below the active threshold value may contribute to the second transitional signal profile.

In normal usage, the device 200 may be in a power off state, such as a sleep state, or other low-power state when the device 200 is in its inactive state. Further, the device 200 may be in a power on state when the device 200 is in its active state, as the user may desire to use the device 200 when the device 200 is in its active state. In operation, the controller 202 may receive and monitor a signal from the biosensor 206. When the signal from the biosensor corresponds to the first transitional state, the controller 202 changes the power state of the device 200 from a power off state to a power on state. In particular, the change in power state from the power off state to the power on state is initiated prior the device 200 entering the active state (i.e., prior to the signal from the biosensor 206 corresponding to the active signal profile). That is, the device 200 is powered on while the user is in the process of putting on the device 200 (i.e., in the first transitional state), rather than in response to the device 200 being mounted on the user's head (i.e., in the active state). Advantageously, the initiation of the change to the power on state while the device 200 is in the first transitional state may reduce a perceived lag or lack of responsiveness of the device 200 by the user once the device 200 reaches the active state.

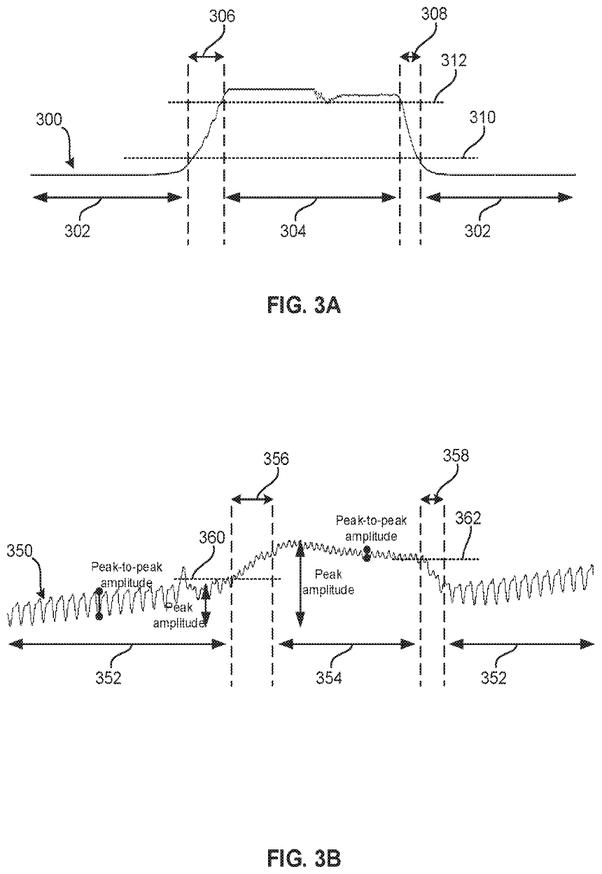

For example, referring to FIGS. 3A and 3B, example signals 300 and 350 detected by biosensors are depicted.

In particular, the signal 300 depicted in FIG. 3A is a PPG signal obtained from a PPG sensor while a user put on and removed a head-mounted device. The signal 300 includes portions 302, 304, 306, and 308 having different profiles. The signal 300 may also be compared to an inactive threshold value 310 and an active threshold value 312.

In the present example, the portion 302 has its peak amplitude below the inactive threshold value 310, and hence corresponds to the inactive signal profile. The portion 304 has its peak amplitude above the active threshold value 312, and hence corresponds to the active signal profile. The portion 306 has its peak amplitude transition from below the inactive threshold value 310 to above the inactive threshold value 310, and hence corresponds to the first transitional signal profile. The portion 308 has its peak amplitude transition from above the active threshold value 312 to below the active threshold value 312, and hence corresponds to the second transitional signal profile.

In some examples, the signal profiles may further be dependent upon a length of time above or below the threshold values 310 and 308. For example, during the portion 304, the signal 300 may briefly drop below the active threshold value 312. To avoid falsely changing the power state to an off state while the user is actively using the device, the second transitional signal profile may define a length of time in which the peak amplitude is below the active threshold value 312 before the signal 300 is determined to correspond with the second transitional signal profile.

The signal 350 depicted in FIG. 3B is an EMG signal obtained from an EMG sensor while a user put on and removed a head-mounted device. The signal 350 includes portions 352, 354, 356, and 358 having different profiles. In the present example, the peak amplitude of the signal 350 may be compared to an inactive threshold value 360 and an active threshold value 362. Further, a peak-to-peak amplitude may be compared to an inactive threshold amplitude and an active threshold amplitude.

In the present example, the portion 352 generally has its peak amplitude below the inactive threshold value 360. In regions where the peak amplitude is above the inactive threshold value 360, the peak-to-peak amplitude is above the active threshold amplitude, and hence the portion 352 is determined to correspond with the inactive signal profile.

The portion 354 generally has its peak amplitude above the active threshold value 362. In regions where the peak amplitude is below the active threshold value, the peak-to-peak amplitude is below the inactive threshold amplitude, and hence the portion 354 is determined to correspond with the active signal profile.

The portion 356 generally has its peak amplitude transition from below the inactive threshold value 360 to above the inactive threshold value 360. Further, the peak-to-peak amplitude is below the active threshold amplitude, and hence the portion 356 is determined to correspond with the first transitional signal profile.

The portion 358 generally has its peak amplitude transition from above the active threshold value 362 to below the active threshold value 362. Further, the peak-to-peak amplitude is above the inactive threshold amplitude, and hence the portion 358 is determined to correspond with the second transitional signal profile.

As will be appreciated, in other examples, other signal profiles may be possible, according to the signals detected by the biosensor. For example, some sensors may produce signals having a peak amplitude above an inactive threshold value when the signal corresponds to the inactive profile, the peak amplitude below an active threshold value when the signal corresponds to an active profile, a peak amplitude transitioning from above the inactive threshold value to below the inactive threshold value when the signal corresponds to the first transitional signal profile, and a peak amplitude transitioning from below the active threshold value to above the active threshold value when the signal corresponds to the second transitional signal profile.

In still further examples, the signal profiles may be based on factors other than peak amplitude threshold values. For example, the signal profiles may be based on a peak-to-peak amplitude of a wave of the signal, or a period or frequency of the wave.

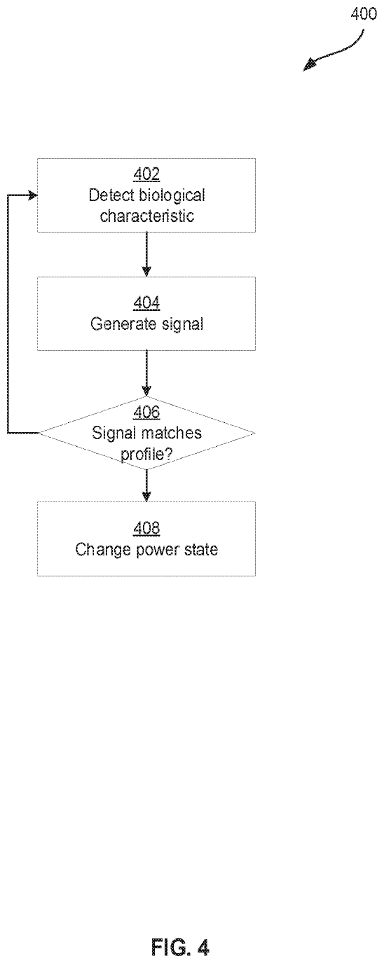

Turning now to FIG. 4, a flowchart of an example method 400 of managing power based on signals from a biosensor. The method 400 will be described in conjunction with its performance by the device 200, and in particular, by the controller 202, as well as the biosensor 206. In other examples, the method 400 may be performed by other suitable devices, such as the device 100.

At block 402, the biosensor 206 detects a biological characteristic of a user. For example, the biosensor 206 may detect a change in blood circulation using a PPG sensor, or electrical activity in facial muscles using an EMG sensor.

At block 404, the biosensor 206 generates a signal representing the biological characteristic of the user. For example, the signal may be similar to a portion of the signal 300 or the signal 350 depicted in FIGS. 3A and 3B. The biosensor 206 may additionally send the signal to the controller 202 for further processing and to manage the power states of the device 200.

At block 406, the controller 202 compares the signal received from the biosensor 206 at block 404 to the signal profiles stored in the memory 204, and in particular, in the signal profile repository 208. In particular, the controller 202 determines whether the signal received from the biosensor 206 at block 404 corresponds to either the first transitional signal profile or the second transitional signal profile.

When the determination at block 406 is negative (i.e., the controller 202 determines that the signal does not correspond to either the first transitional signal profile or the second transitional signal profile), the method 400 returns to block 402, where the biosensor 206 may detect further biological characteristics of the user. In particular, the controller 202 may maintain the current power state of the device 200. For example, if the signal received at block 404 corresponds to the inactive signal profile, the controller 202 may determine that the device 200 is in the inactive state and maintain a low-power or power off state of the device 200 prior to returning to block 402. Similarly, if the signal received at block 404 corresponds to the active signal profile, the controller 202 may determine that the device 200 is in the active state and maintain a power on state of the device 200 prior to returning to block 402.

When the determination at block 406 is affirmative (i.e., the controller 202 determines that the signal corresponds to either the first transitional signal profile or the second transitional signal profile), the method 400 proceeds to block 408. At block 408, the controller 202 changes the power state of the device 200 from a first power state to a second power state according to the transitional signal profile or transitional state of the device 200. In particular, the controller 202 may change the power state from a current power state of the device 200 to an opposing power state of the device 200. For example, if the signal received at block 404 corresponds to the first transitional signal profile, the controller 202 may determine that the device 200 is transitioning from the inactive state to the active state. Accordingly, the controller 202 may change the power state of the device 200 from a low-power or power off state to a power on state. Similarly, if the signal received at block 404 corresponds to the second transitional signal profile, the controller 202 may determine that the device 200 is transitioning from the active state to the inactive state. Accordingly, the controller 202 may change the power state of the device 200 from the power on state to the low-power or power off state.

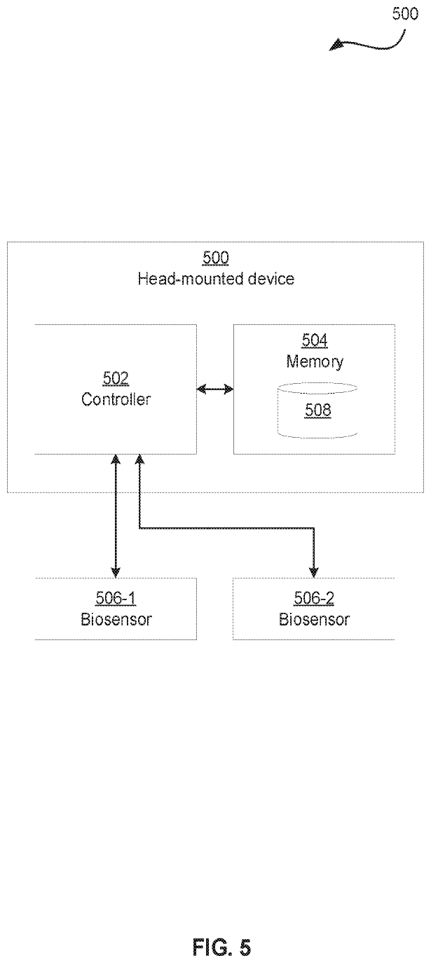

In some examples, the head-mounted device may include or be associated with an additional biosensor to detect an additional biological characteristic of the user and generate an additional signal representing the additional biological characteristic of the user, which may be compared to an additional predefined profile for verifying the activity state of the device. For example, FIG. 5 depicts a block diagram of another example head-mounted device 500. The head-mounted device 500 is similar to the devices 200 and 100 and may be a head-mounted display device, such as an extended reality device, or another suitable head-mounted device. The device 500 includes a controller 502 and a memory 204, and is further associated with biosensors 506-1 and 506-2.

The controller 502 is a power management controller and may include a CPU, a microcontroller, a microprocessor, a processing core, a FPGA, or similar device capable of executing machine-readable instructions. The controller 502 may also include or be interconnected with a non-transitory machine-readable storage medium, such as the memory 504, that may be electronic, magnetic, optical, or other physical storage device that stores executable instructions. In particular, the memory 504 may store an application for managing the power states of the device 500 based on signals from the biosensors 506-1 and 506-2.

The controller 502 is further in communication with the biosensors 506-1 and 506-2. The biosensors 506-1 and 506-2 are sensors which detect biological characteristics of the user and generate signals representing the biological characteristics. Generally, the biosensors 506-1 and 506-2 may detect different biological characteristics. For example, the biosensor 506-1 may be a PPG sensor to detect a change in blood circulation of the user, while the biosensor 506-2 may be an EMG sensor to detect electrical activity of facial muscles of the user. In other examples, the biosensors 506-1 and 506-2 may be other sensors such as eye-tracking sensors, mouth cameras, capacitive sensors, ultrasound sensors, and the like. The biosensors 506-1 and 506-2 may be integrated with the head-mounted device 500 or may be independent from the head-mounted device 500.

The memory 504 further stores a signal profile repository 508 tracking associations between signal profiles of signals detected by the biosensors 506-1 and 506-2 and activity states of the device 500. The activity states of the head-mounted device 500 are similar to the activity states of the device 200 and track a positionally-based state of use of the device 500 rather than power states of the device 500, which track the functional status of the operative components of the device 500. In particular, the signal profile repository 508 may include predefined signal profiles the biosensor 506-1 and additional predefined signal profiles for the biosensor 506-2.

Thus, the signal profile repository 508 may store an active signal profile for the first biosensor 506-1 and an additional active signal profile for the second biosensor 506-2 respectively. Each of the active signal profile and the additional active signal profile may be associated with an active state of the device 500, in which the device 500 is in place on a user's head, in a ready-to-use positional configuration. The signal profile repository 508 may further store an inactive signal profiles and an additional inactive signal profile for the biosensors 506-1 and 506-2 respectively, each corresponding to an inactive state of the device 500, in which the device 500 is removed from a user's head. The signal profile repository 508 further stores a first transitional signal profile and an additional first transitional signal profile for the biosensors 506-1 and 506-2 respectively, each corresponding to a first transitional state, in which the device 500 is being transitioned or moved from the inactive state to the active state. The signal profile repository 508 further stores a second transitional signal profile and an additional second transitional signal profile for the biosensors 506-1 and 506-2 respectively, each corresponding to a second transitional state, in which the device 500 is being transitioned or moved from the active state to the inactive state. As will be appreciated, the signal profiles may be defined by a peak amplitude of the signal relative to a active threshold value and an inactive threshold value, by a peak-to-peak amplitude of the signal, and the like.

In operation, the controller 502 may receive and monitor signals from the biosensors 506-1 and 506-2 and manage the power states of the device 500 based on the signals. In particular, when the signals received from the biosensors 506-1 and 506-2 both correspond to the first transitional state, the controller 502 changes the power state of the device 500 from a power off state to a power on state. Similarly, when the signals received from the biosensors 506-1 and 506-2 both correspond to the second transitional state, the controller 502 changes the power state of the device 500 from the power on state to a low-power or power off state. That is, the activity states determined from the signal profiles of the signals from both the biosensors 506-1 and 506-2 are compared to determine whether or not they match. If they match, the activity state is verified and the controller 502 may change the power state accordingly. The biosensors 506-1 and 506-2 may therefore provide increased accuracy and redundancy to avoid false positive determinations of a transitional state.

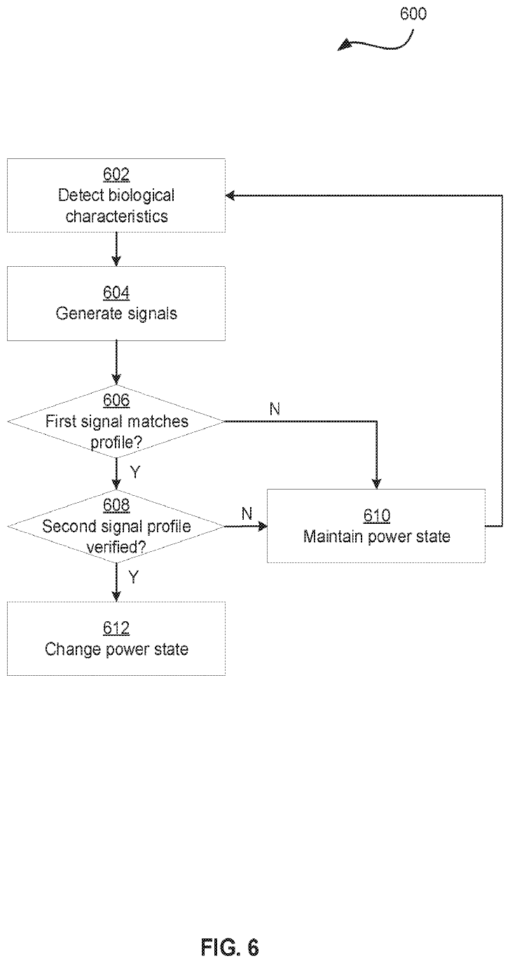

FIG. 6 depicts a flowchart of an example method 600 of managing power based on signals from multiple biosensors. The method 600 will be described in conjunction with its performance by the device 500, and in particular, by the controller 502. In other examples, the method 600 may be performed by other suitable devices.

At block 602, each of the biosensors 506-1 and 506-2 detect respective biological characteristics of a user. For example, the biosensor 506-1 may detect a change in blood circulation using a PPG sensor, and the biosensor 506-2 may detect electrical activity in facial muscles using an EMG sensor.

At block 604, the biosensors 506-1 and 506-2 generate respective signals representing the respective biological characteristics of the user. For example, the signal generated by the biosensor 506-1 may be similar to a portion of the signal 300 depicted in FIG. 3A, while the signal generated by the biosensor 506-2 may be similar to a portion of the signal 350 depicted in FIG. 3B. The biosensors 506-1 and 506-2 may send the respective signals to the controller 502 for further processing and to manage the power states of the device 500.

At block 606, the controller 502 selects the signal from the first biosensor 506-1 received at block 604 and compares it to the signal profiles for the biosensor 506-1. In particular, the controller 502 determines whether the signal received from the biosensor 506-1 at block 604 corresponds to either the first transitional signal profile or the second transitional signal profile for the biosensor 506-1.

When the determination at block 606 is negative (i.e., the controller 502 determines that the signal from the biosensor 506-1 does not correspond to either the first transitional signal profile or the second transitional signal profile for the biosensor 506-1), the method 600 proceeds to block 610. At block 610, the controller 506 maintains the current power state of the device 500. For example, if the signal received at block 604 from the biosensor 506-1 corresponds to the inactive signal profile, the controller 502 determines that the device 500 is in the inactive state and maintains the low-power or power off state of the device 500. Similarly, if the signal received at block 604 from the biosensor 506-2 corresponds to the active signal profile, the controller 502 determines that the device 500 is in the active state and maintains a power on state of the device 500.

When the determination at block 606 is affirmative (i.e., the controller 502 determines that the signal from the biosensor 506-1 corresponds to either first transitional signal profile or the second transitional signal profile for the biosensor 506-1), the method 600 proceeds to block 608. At block 608, the controller 502 makes a similar determination for the additional signal received from the biosensor 506-2 to verify the transitional state of the device 500. That is, the controller 502 selects the additional signal from the second biosensor 506-2 received at block 604 and compares it to the additional signal profiles for the biosensor 506-2. In particular, the controller 502 determines whether the additional signal received from the biosensor 506-2 matches the corresponding additional transitional signal profile for the transitional signal profile detected at block 606. That is, if, at block 606, the controller 502 determines that the signal from the biosensor 506-1 corresponds to the first transitional signal profile for the biosensor 506-1, then at block 608, the controller 502 may verify that the additional signal from the biosensor 506-2 corresponds to the additional first transitional signal profile for the biosensor 506-2. Similarly, if, at block 606, the controller 502 determines that the signal from the biosensor 506-1 corresponds to the second transitional signal profile for the biosensor 506-1, then at block 608, the controller 502 may verify that the additional signal from the biosensor 506-2 corresponds to the additional second transitional signal profile for the biosensor 506-2.

When the determination at block 608 is negative (i.e., the controller 502 determines that the additional signal from the biosensor 506-2 does not correspond to either the respective additional first transitional signal profile or the additional second transitional signal profile for the biosensor 506-2), the method 600 proceeds to block 610. In particular, both the biosensors 506-1 and 506-2 are used to determine that the device 500 is in a transitional state. If either of the biosensors 506-1 or 506-2 fail to detect the transitional state, the current power state is maintained. Accordingly, at block 610, the controller 506 maintains the current power state of the device 500.

When the determination at block 608 is affirmative (i.e., the controller 502 verifies that the signal from the biosensor 506-2 corresponds to the respective transitional signal profile), the method 600 proceeds to block 612. At block 612, the controller 502 changes the power state of the device 500 from a first power state to a second power state according to the transitional signal profile or transitional state of the device 500. In particular, the controller 502 may change the power state from a current power state of the device 500 to an opposing power state of the device 500. For example, if the signals received at block 604 correspond to respective first transitional signal profiles, the controller 502 may determine that the device 500 is transitioning from the inactive state to the active state. Accordingly, the controller 502 may change the power state of the device 500 from a low-power or power off state to a power on state. Similarly, if the signals received at block 604 correspond to the respective second transitional signal profiles, the controller 202 may determine that the device 200 is transitioning from the active state to the inactive state. Accordingly, the controller 202 may change the power state of the device 200 from the power on state to the low-power or power off state.

As described above, an example head-mounted device has a power management controller which controls the power state of the head-mounted device based on signals received from one or more biosensors. In particular, when the signal received from the biosensor the predefined profiles, and, when the signal corresponds to the predefined profile, determine that the head-mounted device is in a transitional state and change the power state of the head-mounted device accordingly. The controller may receive and compare signals from multiple biosensors for accuracy and redundancy, such that the controller changes the power state of the device only when both or all biosensors indicate the transitional state to avoid false positive triggers from any one of the biosensors. Thus, the head-mounted device may conserve power usage by powering off when the head-mounted device is removed. Additionally, since the biosensors allow for detection of the user as the user is putting the head-mounted device on, waking of functional or operational components of the head-mounted device is initiated or complete by the time the head-mounted device is actually on the head of the user. Additionally, the biosensors are not affected by motion or inanimate objects which may otherwise trigger non bio-based presence sensors.

The scope of the claims should not be limited by the above examples, but should be given the broadest interpretation consistent with the description as a whole.

1. A head-mounted device comprising:
a frame to nest the device on a head of a user;
a biosensor disposed on the frame, the biosensor to:
detect a biological characteristic of the user;
generate a signal representing the biological characteristic of the user; and
a power management controller coupled to the biosensor, the controller to, when the signal corresponds to a predefined profile, determine that the head-mounted device is in a transitional state and change a power state of the head-mounted device based on the transitional state.

2. The head-mounted device of claim 1, wherein the biosensor comprises a photoplethysmography sensor to detect a change in blood circulation of the user.

3. The head-mounted device of claim 1, wherein the biosensor comprises an electromyography sensor to detect electrical activity of facial muscles of the user.

4. The head-mounted device of claim 1, wherein the predefined profile is based on a threshold peak amplitude of the signal.

5. The head-mounted device of claim 4, wherein the predefined profile is further based on a threshold amplitude of a wave of the signal.

6. The head-mounted device of claim 1, further comprising an additional biosensor disposed on the frame, the additional biosensor to:
detect an additional biological characteristic of the user; and
generate an additional signal representing the additional biological characteristic of the user; and
wherein the controller is to verify that the head-mounted device is in the transitional state when the signal corresponds to the predefined profile and the additional signal corresponds to an additional predefined profile.

7. A method of power management for a head-mounted device, the method comprising:
detecting, at a biosensor, a biological characteristic of a user;
generating a signal representing the biological characteristic of the user;
when the signal corresponds to a first predefined profile, determining that a head-mounted device is in a first transitional state; and
in response to determining that the head-mounted device is in the first transitional state, changing a power state of the head-mounted device from a first power state to a second power state.

8. The method of claim 7, further comprising:
when the signal corresponds to a second predefined profile, determining that the head-mounted device is in a second transitional state; and
in response to determining that the head-mounted device is in the second transitional state, changing the power state of the head-mounted device from the second power state to the first power state.

9. The method of claim 7, further comprising:
detecting, at an additional biosensor, an additional biological characteristic of the user;
generating an additional signal representing the additional biological characteristic of the user;
when the signal corresponds to the first predefined profile, determining whether the additional signal corresponds to an additional first predefined profile;
when the additional signal corresponds to the additional first predefined profile, verifying that the head-mounted device is in the first transitional state.

10. The method of claim 9, further comprising:
when the additional signal does not correspond to the additional first predefined profile, determining that the head-mounted device is not in the first transitional state; and
maintaining the power state of the head-mounted device.

11. A head-mounted device comprising:
a memory to store, for signals detected by a biosensor associated with the head-mounted device:
an inactive signal profile corresponding to an inactive state of the head-mounted device;
an active signal profile corresponding to an active state of the head-mounted device; and
a first transitional signal profile corresponding to a first transitional state from the inactive state to the active state of the head-mounted device; and
a controller coupled to the memory and the biosensor, the controller to:
receive a signal from the biosensor, the signal representing a biological characteristic of a user; and
when the signal from the biosensor corresponds to the first transitional signal profile, changing a power state of the head-mounted device from a power off state to a power on state.

12. The head-mounted device of claim 11, wherein the controller is to initiate the change of the power state of the head-mounted device from the power off state to the power on state is prior to the signal from the biosensor corresponding to the active signal profile.

13. The head-mounted device of claim 11, wherein
the memory further stores a second transitional signal profile corresponding to a second transitional state from the active state to the inactive state of the head-mounted device; and
wherein the controller is to:
when the signal from the biosensor corresponds to the second transitional signal profile, changing the power state of the head-mounted device from the power on state to the power off state.

14. The head-mounted device of claim 11, wherein the power off state comprises a low-power state.

15. The head-mounted device of claim 11, wherein the biosensor is integrated with the head-mounted device.
